(19) United States
(12) Patent Application Publication
(10) Pub. No.: US 2001/0031949 A1
(43) Pub. Date: Oct. 18, 2001

(54) Protective device for a prefilled injection syringe

(76) Inventor: Hooman A. Asbaghi, Del Mar, CA (US)

Correspondence Address: MATTHEW K. HILLMAN, NYDEGGER & ASSOCIATES, 348 Olive Street, San Diego, CA, 92103, US

(21) Appl. No.: 09/853,294

(22) Filed: May 10, 2001

Related U.S. Application Data
(63) Continuation-in-part of application No. 09/336,405, filed on Jun. 18, 1999.

(51) Int. Cl. A61M 5/00; A61M 5/32

(52) U.S. Cl. 604/198; 604/110

(57) ABSTRACT

A device for covering and protecting the hollow needle of a prefilled injection syringe, after the syringe has been used to inject a fluid medicament into a patient, includes a guard and a guard body. Prior to an injection, the device is installed on the prefilled injection syringe with the guard body affixed to the finger flange of the syringe. During the injection, the guard of the device retracts over the needle and into the guard body to allow the needle to be inserted into the patient. Upon withdrawal of the needle from the patient, the device passively re-covers the needle, locking the guard over the tip of the needle to prevent accidental needlesticks or inadvertent re-use of the syringe.

This application is a continuation-in-part of application Ser. No. 09/336,405 filed Jun. 18, 1999, which is currently pending. The contents of application Ser. No. 09/336,405 are incorporated herein by reference.

### FIELD OF THE INVENTION

The present invention pertains generally to syringes for medical use. More particularly, the present invention pertains to protective devices for prefilled injection syringes. The present invention is particularly, but not exclusively, useful for passively covering and protecting the needle of a prefilled injection syringe after the syringe has been used for an injection.

### BACKGROUND OF THE INVENTION

Recent research from the Centers for Disease Control and Prevention (CDC) shows that approximately 384,000 needle sticks or similar injuries occur among health care workers in U.S. hospitals each year. Unfortunately, each accidental needle stick has the potential to expose a health care worker to a life-threatening virus such as hepatitis or HIV. In addition to the needle sticks that occur in hospitals, accidental needle sticks can also occur in other health care settings. For example, needle stick injuries can occur at clinics or during home health-care. In fact, some studies have estimated that over 600,000 needle sticks occur in the U.S. each year, and approximately 1,000 of these accidental needle sticks result in a life-threatening infection.

For each accidental needle stick, health care providers are obligated to test and counsel the exposed worker. Further, follow-up testing for HIV must be conducted approximately six months after the exposure. It is to be appreciated that the costs associated with the testing, lab work, the workers lost time, and the associated tracking and administrative costs, can be considerable.

Accidental needle sticks can occur in several ways. For example, sudden movement by the patient can cause a health care worker to lose control of a syringe, resulting in injury. Attempts to manually recap a needle after filling the syringe with a medicament or attempts to recap a syringe following an injection can also result in injury. Moreover, injuries often result when contaminated unprotected needles are left unattended or disposed of improperly. In addition to accidental needle sticks, unnecessary exposure to bloodborne pathogens can result when a health care worker mistakenly re-uses a contaminated needle on a patient.

Prefilled injection syringes (i.e. syringes that are delivered to the health care worker containing a single dose of medicament) are commonly used for vaccines, low molecular weight heparins and many of the new biotechnology drugs. By using a prefilled injection syringe several needle handling steps are eliminated for the health care worker, and the risk of inadvertent re-use is lowered. Even with these advantages, the risk of exposure to a used needle is still presented by prefilled injection syringes.

In light of the above, it is an object of the present invention to provide a protective device that is installable on a prefilled injection syringe to passively cover and protect the needle of the syringe after the syringe has been used to inject a medicament into a patient. It is another object of the present invention to provide a protective device for a prefilled injection syringe which allows a needle to be installed on the syringe while the needle remains capped to prevent injury during installation. Still another object of the present invention is to provide a protective device for the capped needle of a prefilled injection syringe that allows for accurately located injections by causing a small portion of the needle to be exposed after the protective device is installed and the cap has been removed. It is yet another object of the present invention to provide a protective device that is installable on a prefilled injection syringe to prevent re-use of a contaminated syringe by locking a guard in place over the contaminated needle. Yet another object of the present invention is to provide a protective device for a prefilled injection syringe that is easy to use, relatively simple to implement, and comparatively cost effective.

### SUMMARY OF THE PREFERRED EMBODIMENTS

A device for covering and protecting the hollow needle of a prefilled injection syringe, after the syringe has been used to inject a fluid medicament into a patient, includes a guard, a guard body and a coil spring. Prior to an injection, the device is installed on the prefilled injection syringe and affixed to the finger flange of the syringe. During the injection, the guard of the device retracts over the needle allowing the needle to be inserted into the patient. Upon withdrawal of the needle from the patient, the device passively re-covers the needle, locking the guard over the tip of the needle to prevent accidental needlesticks or inadvertent re-use of the syringe.

For the present invention, the guard body is formed as a hollow cylinder that is large enough to slide over the hollow needle and syringe barrel of the prefilled syringe. A flat is formed at the proximal end of the guard body to seat the guard body against the finger flange of the prefilled syringe. Clips extend from the guard body to hold and lock the guard body against the finger flange of the prefilled syringe. Once the syringe and guard body are locked together, the finger flange of the prefilled syringe becomes covered by the guard body and no longer functions as a finger flange. Consequently, the guard body is formed with a replacement finger flange at the proximal end of the guard body.

In detail, like the guard body, the guard is formed as a hollow cylinder defining a longitudinal axis. As such, a cylindrical aperture extends through the guard. This allows a needle that is covered by a standard needle cap to be passed through the aperture of the guard. Importantly, the cylindrical guard is sized to allow it to be inserted into the distal end of the guard body. With this combination of structure, the guard body is disposed over the guard allowing reciprocal movement between the guard and guard body. Additionally, a biasing mechanism, such as a spring, is mounted between the guard and the guard body to urge the guard in a distal direction, relative to the guard body.

An important aspect of the present invention involves the cooperative interaction between the guard and the guard body. To effect this interaction, a plug extends from the guard and into a linear slot that is formed on the guard body. In detail, this linear slot is aligned substantially parallel to the longitudinal axis of the guard body and is formed with a proximal end and a distal end. A latching cut out is formed between the two ends and a locking cut out is formed at the distal end.

In operation, the device is initially configured with the plug of the guard located in the slot of the guard body at the latching cut out position between the ends of the slot. Next, the capped needle of the prefilled syringe is inserted into the proximal end of the guard body and passed through the aperture of the guard. The insertion of the syringe into the protective device is continued until the finger flange of the syringe contacts the flat formed in the guard body. At this point, the clips formed on the guard body lock the syringe to the guard body. Also, after the syringe has been inserted into the protective device as described above, a relatively short portion of the needle extends distally from the guard. This small extension allows the needle tip to be accurately located on the patient for the injection. The syringe is now ready for injecting the fluid medicament into the patient.

To perform an injection, the needle is pushed into the body of the patient until the guard contacts the patient. During contact between the guard and the patient, movement of the guard is stopped. Meanwhile, the syringe and guard body can be further translated towards the patient, inserting the needle to the proper penetration depth. Thus, the guard body moves relative to the guard during insertion of the needle. During this movement, the plug is directed from the latching cutout, into the linear track and towards the proximal end of the linear track. Fluid medicament can then be injected into the patient by the syringe.

After injection of the medicament, the needle is withdrawn from the patient. During the withdrawal of the needle, the syringe and guard body are pulled away from the patient while the guard remains in contact with the body of the patient. Specifically, the spring expands to hold the guard against the patient. Also, during the withdrawal of the needle, the spring expands to translate the plug along the linear slot towards the distal end of the linear slot.

After the needle is completely withdrawn from the patient, the syringe including the needle are pulled away from the body of the patient. During this movement, the spring continues to expand causing the guard and plug to move distally until the plug reaches the distal end of the slot. When the distal end of the slot is reached, the locking cutout (detent) formed at the distal end of the slot prevents further axial movement of the plug relative to the slot. Stated differently, the locking cutout effectively locks the guard to the guard body and over the hollow needle. Importantly, in this configuration, the guard completely covers the hollow needle to protect against accidental needle sticks or inadvertent re-use.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a protective device in accordance with the present invention installed on a prefilled injection syringe;

FIG. 2 is an exploded sectional view of a protective device in accordance with the present invention together with a prefilled injection syringe as seen along line 2-2 in FIG. 1;

FIG. 3 is a sectional view (enlarged for clarity) of the proximal end of a guard as seen along line 3-3 in FIG. 2 showing a pair of tabs that extend radially to limit rotation of the guard relative to the guard body after the completion of an injection;

FIG. 4 is a sectional view (enlarged for clarity) of the distal end of a guard body as seen along line 4-4 in FIG. 2 showing a pair of grooves formed in the inner wall of the guard body that interact with the tabs of the guard to limit rotation of the guard relative to the guard body after the completion of an injection;

FIG. 5A is a plan view of a protective device in accordance with the present invention after installation onto a prefilled injection syringe having a capped needle;

FIG. 5B is a plan view of a protective device as shown in FIG. 5A after removal of the needle cap;

FIG. 5C is a plan view of a protective device as shown in FIG. 5B after the needle of the prefilled syringe has been inserted into the body of a patient to the proper depth for an injection; and

FIG. 5D is a plan view of a protective device as shown in FIG. 5C after the needle of the prefilled syringe has been withdrawn from the body of a patient.

### DESCRIPTION OF THE PREFERRED EMBODIMENT

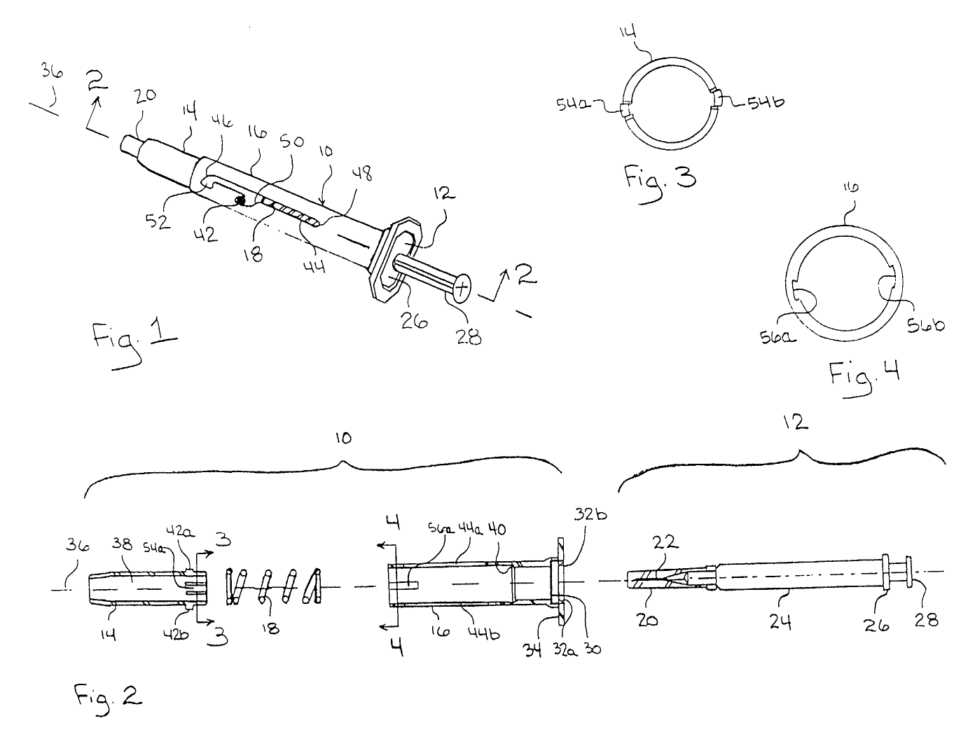

Referring initially to FIG. 1, a protective device 10 in accordance with the present invention is shown installed on a prefilled injection syringe 12. As shown, the device 10 includes a guard 14, a guard body 16 and a coil spring 18. With cross reference to FIGS. 1 and 2, it can be seen that the device 10 can be installed onto the prefilled injection syringe 12 while the cap 20 of the syringe 12 remains in place.

Referring now to FIG. 2, an exemplary prefilled injection syringe 12 for use with the present invention is shown. As shown, the prefilled syringe 12 includes a hollow needle 22 mounted on a syringe barrel 24. A finger flange 26 is formed at the proximal end of the syringe 12 to aid in depressing the plunger 28 of the syringe 12. Further, it is to be appreciated that the prefilled syringe 12 includes a single dose of medicament that is generally placed in the syringe 12 prior to delivering the syringe 12 to the point of use. Further, needle 22 of the prefilled syringe 12 is generally covered with a protective cap 20 immediately after the filling of the syringe 12 with medicament to protect the needle 22 during handling prior to use.

Referring still to FIG. 2, it can be seen that the guard body 16 is formed with as a hollow cylinder, open at both its proximal and distal ends. For the present invention, as shown, the guard body 16 is sized large enough to be slid over the hollow needle 22 and syringe barrel 24 of the prefilled syringe 12. As shown, a flat 30 is formed at the proximal end of the guard body 16 to allow the finger flange 26 of the prefilled syringe 12 to seat against the guard body 16. Clips 32a, b extend from the guard body 16 to hold and lock the guard body 16 against the finger flange 26 of the prefilled syringe 12 when the device 10 is installed on the prefilled syringe 12. As shown, a beveled surface on each clip 32a, b allows the finger flange 26 to pass the clips 32a, b and a slot on the flat 30. As further shown, a replacement finger flange 34 is formed at the proximal end of the guard body 16 to aid in depressing the plunger 28 during an injection. As shown in FIG. 1, once the syringe 12 and guard body 16 are locked together, the finger flange 26 of the prefilled syringe 12 is no longer functional as a finger flange.

Referring back to FIG. 2, it can be seen that the guard 14 is also formed as a hollow cylinder defining a longitudinal axis 36. As shown, a substantially cylindrical aperture 38 extends through the guard 14 sized large enough to allow the cap 20 of the syringe 12 to be passed through the aperture 38. Preferably, the distal end of the guard 14 is tapered, as shown. Importantly, the cylindrical guard 14 is sized to allow the guard 14 to be inserted into the distal end of the guard body 16. With this combination of structure, the guard body 16 can be disposed over the guard 14 allowing reciprocal movement between the guard 14 and guard body 16, as shown in FIG. 1.

Referring back to FIG. 2, a coil spring 18 is provided for the device 10 to urge the guard 14 in a distal direction, relative to the guard body 16. As shown, the spring 18 is sized to allow for insertion of the spring 18 into the distal end of the guard body 16. A ledge 40 is formed in the guard body 16 to seat the proximal end of the spring 18. As shown, the distal end of the spring 18 seats against the edge of the guard at the proximal end of the guard 14.

Referring now with cross reference to FIGS. 1 and 2, it can be seen that the guard 14 is formed with a pair of substantially identical plugs 42a, b extending outwardly from the axis 36. Further, the guard body 16 is formed with a pair of substantially identical linear slots 44a, b that each extend through the cylindrical wall of the guard body 16. As shown, the guard 14 can be disposed within the guard body 16, with each plug 42 inserted into a respective slot 44. Specifically, the distal end of the guard can be deformed slightly and then pressed into the proximal end of the guard body. As shown, each linear slot 44 is aligned substantially parallel to the longitudinal axis 36 and is formed with a proximal end 46 and a distal end 48. Further, each slot 44 is formed with a latching cutout 50 positioned between the proximal end 46 and the distal end 48, and a locking cutout 52 positioned at the distal end 48.

Referring now with cross reference to FIGS. 2 and 3, it can be seen that the proximal end of a guard 14 is formed with a pair of tabs 54a, b that extend outwardly from the axis 36. Further, with cross reference to FIGS. 2 and 4, it can be seen that the distal end of the guard body 16 is formed with a pair of grooves 56a, b that extend into the inner wall of the cylindrical guard body 16. The tabs 54 and grooves 56 are positioned on the guard 14 and guard body 16 so that when the plugs 42 of the guard 14 are positioned in the locking cutouts 52 of the slots 44, the tabs 54 will each be inserted into a respective groove 56. It is to be appreciated that when the tabs 54 are inserted into the grooves 56 they will limit rotation of the guard 14 relative to the guard body 16.

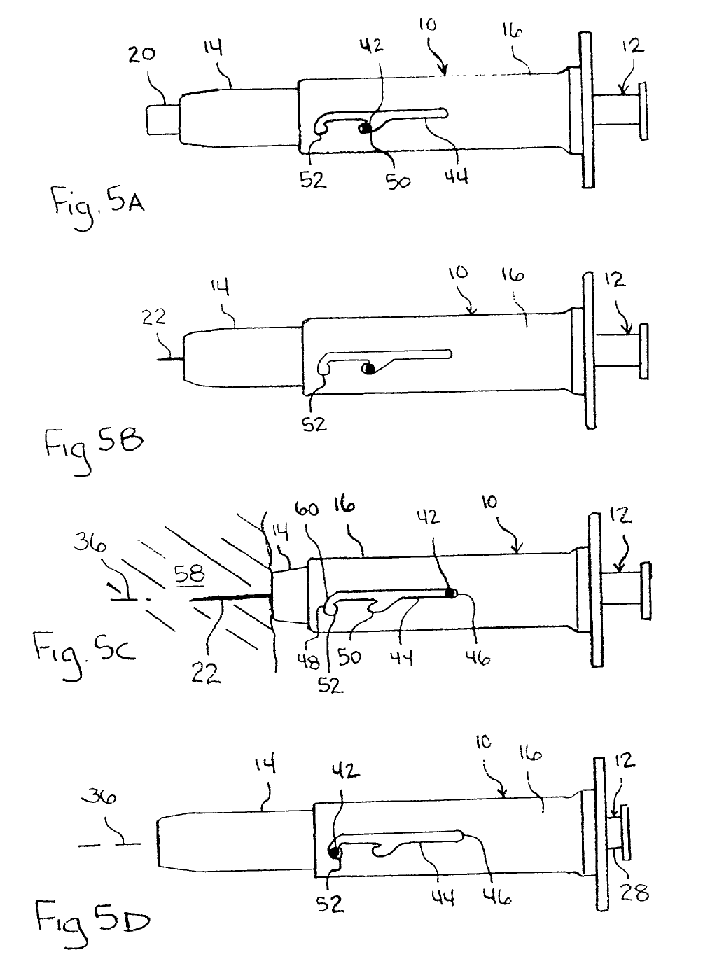

The operation of the device 10 can best be appreciated with reference to FIGS. 5A-D. FIG. 5A shows that in accordance with the present invention, the device 10 is initially configured with the plugs 42 of the guard 14 located in the slots 44 of the guard body 16 and positioned at the latching cutout 50. While the device 10 is in this configuration, the distal end of the prefilled syringe 12 (with cap 20 installed) is inserted into the proximal end of the guard body 16 until the cap 20 extends distally from the guard 14. FIG. 5B shows the device 10 installed on the syringe 12 after removal of the cap 20. As shown, after removal of the cap 20, a small portion (tip) of needle 22 extends distally from the guard 14. This small extension allows the tip of the needle 22 to be accurately located on the patient for the injection.

Next, as shown in FIG. 5C, the needle 22 is inserted into the body of the patient 58 until the guard 14 contacts the patient 58. The syringe 12 and guard body 16 can be further translated towards the patient 58, inserting the needle 22 to the proper penetration depth. Thus, the guard body 16 moves relative to the guard 14 during insertion of the needle 22 into the patient 58. By cross referencing FIGS. 5B and 5C it can be seen that during the penetration of the patient 58 with the needle 22, the plugs 42 are directed from the latching cutouts 50, into the linear track of the slots 44 and towards the proximal end 46 of each linear track. Once the needle 22 is inserted into the patient 58 at the proper depth, fluid medicament can be injected into the patient 58 by depressing the plunger 28 of the syringe 12.

With cross reference to FIGS. 5C and 5D, it can be seen that during the withdrawal of the needle 22 from the patient 58, the needle 22, guard body 16 and syringe 12 move in a proximal direction relative to the guard 14. Specifically, the spring 18 (shown in FIG. 2) expands to hold the guard 14 against the patient 58 while the needle 22 is withdrawn. During the withdrawal of the needle 22 from the patient 58, the spring 18 (shown in FIG. 2) expands to translate the plugs 42 along the linear slots 44 towards the distal end 48 of the linear slots 44.

After the needle 22 is completely withdrawn from the patient 58, the syringe 12 and the needle 22 are pulled away from the body of the patient 58. During this movement, the spring 18 (shown in FIG. 2) continues to expand causing the guard 14 and plugs 42 to move distally until the plugs 42a, b reach the distal end 48 of each slot 44a, b. At the distal end 48 of the slots 44, a guide ramp 60 formed in each slot 44 causes the plug 42 to move azimuthally (with respect to the axis 36) and axially into the locking cutout 52. Thus, the guard 14 rotates relative to the guard body 16 as the plugs 42 move from the linear portion of each slot 44 into the locking cutout 52. This rotation allows the tabs 54 (shown in FIG. 3) formed in the proximal end of the guard 14 to insert into the grooves 56 (shown in FIG. 4) formed in the distal end of the guard body 16. Accordingly, when the plug 42 is in the locking cutout 52, as shown in FIG. 5D, axial movement of the guard 14 relative to guard body 16 and syringe 12 is blocked by the locking cutout 52 while rotational movement of the guard 14 relative to guard body 16 and syringe 12 is blocked by the interaction of the tabs 54 and grooves 56. Importantly, in this configuration, the guard 14 is locked over the hollow needle 22 of the syringe 12 to protect against accidental needle sticks or inadvertent re-use.

While the particular devices and methods as herein shown and disclosed in detail are fully capable of obtaining the objects and providing the advantages herein before stated, it is to be understood that they are merely illustrative of the presently preferred embodiments of the invention and that no limitations are intended to the details of construction or design herein shown other than as described in the appended claims.

What is claimed is:

1. A device for protecting the hollow needle of a prefilled injection syringe, said device comprising:
a hollow cylindrical guard defining an axis and formed with at least one plug extending radially outwardly therefrom;
a guard body disposed on said guard for reciprocal movement thereon, said guard body having a means for affixing said guard body to the prefilled injection syringe with said needle extending through said guard, said guard body being formed with a linear slot aligned substantially parallel to said axis with said plug inserted into said slot for movement therein, said slot having a proximal end and a distal end with a latching cut out formed therebetween and a locking cut out formed at said distal end; and
a biasing means disposed between said guard and said guard body to urge said guard and said guard body in opposite axial directions and, in sequence, to initially hold said plug in said latching cut out to partially extend said needle from said guard, to then allow said plug to move in said slot toward said proximal end to further extend said needle from said guard in response to an external force against said guard, and to subsequently move said plug into said locking cut out at said distal end to cover and protect said needle with said guard upon removal of the external force.

2. A device as recited in claim 1 wherein said biasing means is a spring.

3. A device as recited in claim 1 wherein a first length of said needle extends from said guard when said plug is held in said latching cut out, and a second length of said needle extends from said guard when said plug is moved into said slot and toward said proximal end, with said second length being greater than said first length.

4. A device as recited in claim 1 wherein said plug extends is shaped as a solid cylinder.

5. A device as recited in claim 1 wherein said guard body is formed with two said slots and said guard is formed with two said plugs with each said plug inserted into a respective said slot.

6. A device as recited in claim 1 wherein said prefilled injection syringe includes a needle cap for protecting the needle before injection, and wherein said guard is sized to allow said needle cap to pass therethrough.

7. A device as recited in claim 1 wherein said prefilled injection syringe includes at least one finger flange, and wherein said means for affixing said guard body to the prefilled injection syringe comprises a clip formed at the proximal end of said guard body for affixing said guard body to said finger flange.

8. A device as recited in claim 1 wherein said guard body is substantially cylindrically shaped.

9. A device as recited in claim 8 wherein said guard body is substantially cylindrically shaped and formed with a finger flange at the proximal end of said guard body.

10. A device for protecting the hollow needle of a prefilled injection syringe after the syringe has been used to inject a patient, said device comprising:
a substantially cylindrically shaped guard defining an axis, said guard formed with an aperture for receiving the hollow needle therethrough;
a guard body disposed on said guard for axial movement thereon, said guard body having a means for affixing said guard body to the prefilled injection syringe;
a means for biasing said guard along said axis in a distal direction from said guard body; and
a means for interconnecting said guard with said guard body for initially holding said guard in a first position relative to said guard body, in response to said biasing means, and for sequentially directing said guard through a second position and into a needle-protecting third position relative said guard body in response to a selectively applied force against said guard in opposition to said biasing means.

11. A device as recited in claim 10 wherein said biasing means is a coil spring.

12. A device as recited in claim 10 wherein said interconnecting means comprises:
a plug formed on said guard and extending therefrom; and
a slot formed in said guard body for engagement with said plug.

13. A device as recited in claim 12 wherein said slot is formed with a slot portion that is substantially linear and substantially parallel to said axis, said slot portion having a proximal end for contact with said plug when said guard is in said second position, and a distal end formed with a lockout for contact with said plug when said guard is in said third position.

14. A device as recited in claim 10 wherein said prefilled injection syringe includes a needle cap for protecting the needle before injection, and wherein said aperture formed in said guard is sized to allow said needle cap to pass therethrough.

15. A device as recited in claim 10 wherein said prefilled injection syringe includes at least one finger flange, and wherein said means for affixing said guard body to the prefilled injection syringe comprises a clip formed at the proximal end of said guard body for affixing said guard body to said finger flange.

16. A device as recited in claim 10 wherein said guard body is substantially cylindrically shaped and formed with a finger flange at the proximal end of said guard body.

17. A device as recited in claim 10 wherein said guard body is formed with a groove and said guard is formed with a tab for insertion into said groove when said guard is in said needle-protecting third position to prevent rotation of said guard relative to said guard body.

18. A method for injecting a medicament into a patient using a prefilled syringe and passively protecting the used syringe thereafter, said method comprising the steps of:
providing a prefilled syringe having a hollow needle defining an axis;
providing a spring, a guard formed with an aperture and at least one plug extending from said guard, and a guard body formed with a linear slot having a proximal end and a distal end with a latching cut out formed therebetween and a locking cut out formed at said distal end;
disposing said guard body on said guard for reciprocal movement thereon with said spring interposed therebetween to urge said guard and said guard body in opposite axial directions;
positioning said plug in said latching cut out;
inserting said needle through said aperture of said guard;
affixing said guard body to said prefilled syringe;
pressing said hollow needle into the patient to first establish contact between said guard and the patient and then to retract said guard over said needle;
injecting medicament into the patient; and
removing said needle from the patient to allow said biasing means to relocate said plug along said slot and into said lockout to cover and protect said needle with said guard.

19. A method as recited in claim 18 wherein said prefilled syringe is formed with a finger flange and said affixing step is accomplished by clipping said guard body onto said finger flange of said prefilled syringe.

20. A method as recited in claim 18 wherein said prefilled syringe includes a needle cap positioned over said needle and said needle cap is inserting through said aperture of said guard with said needle during said insertion step and wherein said method further comprises the step of removing said needle cap from said needle after said insertion step.
